(19) United States
(12) Patent Application Publication
(10) Pub. No.: US 2025/0229083 A1
(43) Pub. Date: Jul. 17, 2025

(54) TUMOR TREATING SYSTEMS

(71) Applicant: LifeBridge Innovations, PBC, Longwood, FL (US)

(72) Inventor: Peter F. Travers, Longwood, FL (US)

(73) Assignee: LifeBridge Innovations, PBC, Longwood, FL (US)

(21) Appl. No.: 19/013,869

(22) Filed: Jan. 8, 2025

Related U.S. Application Data
(60) Provisional application No. 63/619,968, filed on Jan. 11, 2024.

(51) Int. Cl. A61N 1/36; A61N 1/04; G16H 20/40

(52) U.S. Cl. CPC A61N 1/36002; A61N 1/0408; A61N 1/0476; A61N 1/36034; G16H 20/40

(57) ABSTRACT

A method of adapting a treating of tumors by the delivery of tumor treating electromagnetic fields to a patient including the steps of: placing an electrode array on the patient; selecting a first adaptation of an electrode activation configuration to activate selected elements of the electrode array to treat the tumors; analyzing cells of the tumors that have been treated using the first adaptation of the electrode activation configuration; and using the first adaptation of the electrode activation configuration to treat the tumors until depending on a cell analysis of the analyzing step then changing to a second adaptation of the electrode activation configuration to activate selected elements of the electrode array to treat the tumors, the first adaptation including a rotating of electromagnetic field frequencies between 200 kHz and 150 kHz.

### CROSS REFERENCE TO RELATED APPLICATIONS

This is a non-provisional patent application based upon U.S. provisional patent application serial no. 63/619,968, entitled “TUMOR TREATING SYSTEMS”, filed Jan. 11, 2024.

### BACKGROUND OF THE INVENTION

### 1. Field of the Invention

The present invention relates to tumor and cancer cell treatment and more specifically to treatments involving the application of electromagnetic fields.

### 2. Description of the Related Art

Alternating Electric Fields, also referred to as Tumor Treating Fields (TTFs or TTFields), can be employed as a type of cancer treatment therapy by using low-intensity electromagnetic fields. These low-intensity fields rapidly change direction, thousands of times per second. Since the TTFs are electric fields, they do not cause muscle twitching or severe adverse side effects on other electrically activated tissues. The growth rate of cancer cells is typically greater than the growth rate of normal, healthy cells. Alternating Electric Fields therapy takes advantage of this high growth-rate characteristic. TTFs act to disrupt a cancer cell's mitotic process and cytokinesis by manipulating the cell's polarizable intracellular constituents, namely tubulins that form mitotic spindles that pull the genetic material in the nucleus into two sister cells. Tubulins form mitotic spindles by taking on electrical properties called dipole moments, which is tubulin molecules become positively charged on one side and negatively charged on the other side. Tubulin form mitotic spindles by connecting to each other positive to negative forming chains. TTFs interrupt mitotic spindle microtubule assembly by interfering with the electric bonds between tubulin molecules thereby preventing cell division. The metastatic disease cells treated using TTFs will go into programmed cell death usually within 4 to 5 hours. Those cancer cells that do manage to divide create malformed daughter cells that are recognized by the immune system as foreign and are thereby attacked. The result is a significant reduction in tumor size and potential for full elimination of solid tumors. TTFs are tuned to treat specific cancer cells and thereby do not damage normal cells. TTF therapy can be used as a sole treatment method, or it can be combined with conventional drug delivery mechanisms.

TTFs are applied to patients using insulated electrodes adhered to the skin by a variety of methods including the use of medical adhesives, articles of clothing, etc. There are multiple configurations of insulated electrodes, but all have an insulated material with a high dielectric constant on one side and a thin metal coating on the other, usually silver. Prior art insulated electrodes used to generate TTFs always come in pairs.

It is well established that delivering TTF therapy from more than one angle increases tumor reduction. This is because of a phenomenon called dielectrophoresis. During the telophase of mitosis, a cleavage furrow forms between the emerging daughter cells. When a TTField runs parallel through the cleavage furrow Polarizable objects are pulled toward the highest concentration of electric field (now the cleavage furrow), in this case, the genetic material needed for cell division. This bulk of material packing the cleavage furrow causes it to burst, causing cell death. This bursting of targeted cancer cells leads to increased tumor reduction in addition to the base mechanism of action of TTFs. Since cancer cells divide in random positions and orientations a TTF from one direction miss opportunities to cause dielectrophoresis. Prior art TTF systems use fixed arrays that presently only deliver therapy from 2 angles.

The success of TTF therapy is also dependent on intensity. TTFields of 1 V/cm may slow a tumor down but still leave more cells growing than are being killed. However, intensities producing TTFields of 2.35 V/cm kill more cells than grow, potentially fully defeating a Tumor. The problem that intensity brings to successful TTF therapy is heat management. Prior art reacts to rising temperatures caused by increased intensity by shutting down the entire TTF system for cool down periods when array temperatures reach 105.5 F. This reactive approach to heat management has been observed to only deliver therapy to a Patient 39% of the time the device is being worn in some cases. These extended shutdowns reduce the efficacy of TTF therapy.

What is needed in the art, is a TTF system that adaptively treats tumors.

### SUMMARY OF THE INVENTION

The present invention provides an improved cancer and tumor treatment regime utilizing TTF therapy.

The invention in one form is directed to a method of adapting a treating of tumors by the delivery of tumor treating electromagnetic fields to a patient including the steps of: placing an electrode array on the patient; selecting a first adaptation of an electrode activation configuration to activate selected elements of the electrode array to treat the tumors; analyzing cells of the tumors that have been treated using the first adaptation of the electrode activation configuration; and using the first adaptation of the electrode activation configuration to treat the tumors until depending on a cell analysis of the analyzing step then changing to a second adaptation of the electrode activation configuration to activate selected elements of the electrode array to treat the tumors, the first adaptation including a rotating of electromagnetic field frequencies between 200 kHz and 150 kHz. A rotating of electromagnetic field frequencies takes place within a range of frequencies of between 200 kHz and 150 kHz.

The invention in another form is directed to a method of treating of tumors by the delivery of tumor treating electromagnetic fields to a patient including the steps of: placing at least one electrode array on the patient; selecting a first adaptation of an electrode activation configuration to activate selected elements of the electrode array to treat the tumors; analyzing cells of the tumors that have been treated using the first adaptation of the electrode activation configuration; and using the first adaptation of the electrode activation configuration to treat the tumors until, depending on a cell analysis of the analyzing step, then changing to a second adaptation of the electrode activation configuration to activate selected elements of the electrode array to treat the tumors.

The invention is yet another form is directed to a method of method of treating tumors by the delivery of tumor treating electric fields to a patient including the steps of: importing a medical image of the patient into a medical simulator; placing at least one electrode array on the patient; using the medical simulator to simulate a firing configuration of the electrodes to be included in adaptations of the electrode activation configuration; selecting a first adaptation of an electrode activation configuration to activate selected elements of the electrode array to treat the tumors, the first adaptation of an electrode activation configuration including the firing configuration of a first adaptation of the electrode activation configuration; analyzing cells of the tumors that have been treated using the first adaptation of the electrode activation configuration; and using the first adaptation of the electrode activation configuration to treat the tumors until depending on a cell analysis of the analyzing step then changing to a second adaptation of the electrode activation configuration to activate selected elements of the electrode array to treat the tumors.

Advantageously the present invention includes the adaptations of alternate treatments during the tumor treating regime.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 illustrates, in a schematic form, a tumor treating system of the present invention that utilizes TTFs;

FIG. 2A is the first page of a flowchart illustrating an embodiment of a tumor treatment method of the present invention;

FIG. 2B is the second page of the flowchart of FIG. 2A;

FIG. 2C is the third page of the flowchart of FIGS. 2A and 2B;

FIG. 3 illustrates another method of treating tumors including the use of a simulator to predict where cancer is likely to next start and determine treatments;

FIG. 4 illustrates an embodiment of the present invention with an electrode configuration of three isolated, in this case concentric, electrodes;

FIG. 5 illustrates yet another embodiment of the present invention with a configuration of four electrodes of FIG. 4 arranged in a pattern with a central controller;

FIG. 6 is a schematical cross-sectional view of a patient's body with tumors located therein;

FIG. 7 is another view of the patient's body of FIG. 6, with illustrated firings of tumor treating fields at various angles, some using the same electrodes;

FIG. 8 is a chart illustrating the effectiveness of using an embodiment of the present invention at selected angles;

FIG. 9A is the first page of a flow chart illustrating the embodiment of a tumor treatment method of the present invention that alters the treatment regime responsive to the treatment progress; and

FIG. 9B is the second page of the flow chart of FIG. 9A.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

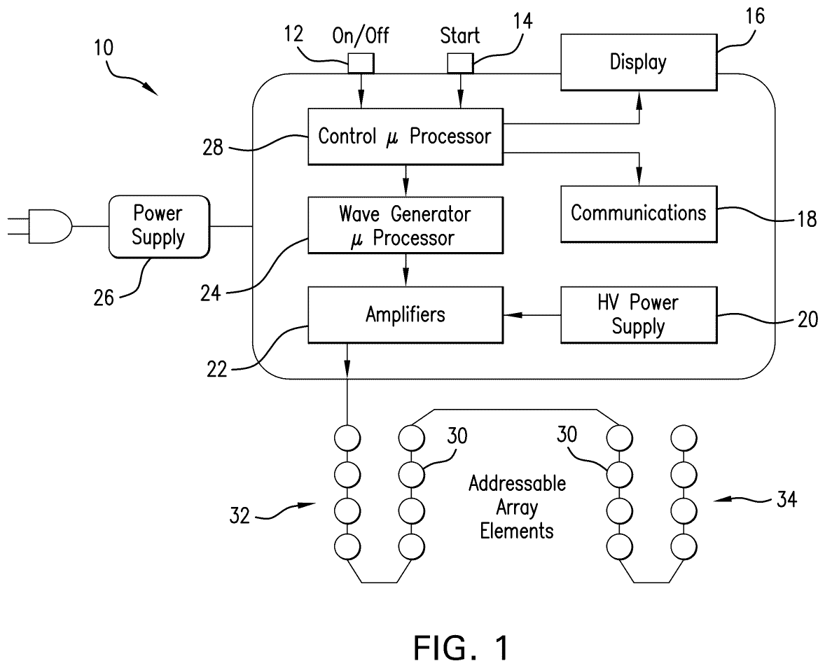

Referring to the drawings and more particularly to FIG. 1 there is illustrated in schematic form a tumor treating apparatus 10 used by the various embodiments of the present invention.

Applicant's inventive systems include make use of tumor treating apparatus 10 having a power on/off selector 12, a start process selector 14, a display 16, a communications device 18, a high voltage power supply 20, amplifiers 22, a wave generator 24 with a micro-processor, a power supply 26, a control device 28 with a micro-processor, electrodes 30 arranged in a first array 32 and a second array 34. The present invention may use removable application racks for holding Tumor Treating Field (TTF) electrodes/transducers 30, arranged in arrays 32 and 34.

Transducers 30 in arrays 32, 34, that deliver tumor treating fields through a patient's body to reduce solid tumors, are held in place on the patient by a water soluble, conductive medical grade adhesive applied to the under-side of each transducer 30. In preparation for the application of the arrays on a patient's body, transducer arrays are placed in an application tool (or application rack). The tool consists of flexible racks that hold the arrays in the needed patient body shape and electrode positioning for adhering the transducers to the body in selected locations. The conductive medical adhesive is applied to the arrays while the arrays are in the flexible removable racks. The flexible racks are used to press the electrodes 30 of the arrays 32, 34 onto a patient's skin in the predetermined position allowing the medical adhesive to adhere. Once electrodes 30 of the arrays 32, 34 are held in place by the medical adhesive the flexible racks are removed prior to the beginning of therapy. In the present invention the transducer arrays 32, 34 on a patient is built in subsections and then linked together as one larger array, many times covering the entire torso. The large arrays consist of software-controlled array elements 30 (electrodes). Through software control the array elements are dynamically divided into sub arrays that are selected to make up the optimal firing configuration.

In one embodiment transducer arrays 32, 34 are fit into the flexible rack in such a way as to be flush with the bottom portion of each array element and to form a seal around each one. Then a masking material is placed over the bottom of the array covering the entire flexible rack but leaving the bottom of each array element 30 exposed. The masking material is held in place through multiple connection points on the flexible racks. Then the medical adhesive is applied to the bottom of electrodes 30 of the arrays 32, 34 via a spraying device or manual squidgy, brush or other application tool. Once the medical adhesive is applied the masking material is unfastened from the flexible rack and removed. The seal around each array element in the flexible rack keeps the medical adhesive from migrating to the side of the arrays. The medical adhesive is allowed to air dry long enough to become tacky. With the array 32, 34 still in the flexible rack the array 32, 34 is held in the proper position on the patient and then gently but firmly pressed onto the patient. Once the medical adhesive is holding elements 30 of the arrays in place on the patient's body the flexible rack is removed by peeling it from the array 32, 34.

In another embodiment of the present invention the flexible removable racks are color coded to help guide correct placement. Patients receive semi-permanent-colored tattoos. The tattoos can be in the form of colored dots or other shapes. The tattoos can also be symbols indicating the direction of array placement (top, bottom, left, right, etc.). Once ready the flexible racks are lined up with corresponding colors and symbols for accurate placement of electrodes 30. For example, blue might be upper left back to be placed by symbols (left side here, etc.).

In another embodiment of the present invention of the flexible removable racks are marked with reference points for skeletal indexing, to ensure proper placement. In many cases the placement of arrays are consistently placed in the same place on the body through skeletal indexing. For example, the upper left back subsection array may be placed in a flexible removable rack that has a mark on the 3^{rd} row right side indicating C7, meaning the C7 mark should be placed in line with the Cervical vertebrae number 7. This system of marks can be used in a wide variety of skeletal indexes.

In another embodiment the flexible removable racks have indicators that ensure that arrays are placed in the rack in the correct order and held up to the body in the proper position (not upside down, etc.). Array subsections are equipped with connectors on each end, one female and the other male. The connectors are used to link one subsection to the next to form one large array ready to be divided into programed subsections, regardless of what subsection an array element is a part of. The software does not see subsections, but only one large array made up of array elements.

If an array subsection is placed into a flexible removable rack upside down, the placement marking on the rack would cause the array subsection to be placed accordingly. The result would be a male connector being positioned to connect with another male which is not possible. This prevents such an error from occurring, which would cause an inconvenient redo and a delay in therapy.

To prevent this incorrect placement of subsections in an application tool (flexible rack), safeguards are built into the design of the tool. Not only do the flexible racks have visible placement directions (Male connector end starts here, etc.), the tool also has simulated connectors extending from each end.

Although the application tools can take on any shape, they are mostly rectangles made up of rows and columns. On the top row of the application tool extends out a simulated female connector, on the bottom extends out a simulated male connector. The first step in placing a flexible removable rack is to plug its female connector into the simulated male connector. The female connector will only go into a male connector forcing a correct placement before maintenance and medical adhesive application. The simulated connectors are also designed to act as waterproof protectors of the actual connectors which can then be subject to water spraying for cleaning after a therapy session.

In another embodiment the flexible racks that make up the application tool are designed to allow the arrays to morph into unusual shapes. In many patients it is difficult to place transducer arrays on their bodies because of skin issues. In some cases, patients may have scars from surgery. In another case they may have fat rolls creating voids where skin contact cannot be maintained. In another case a female may have large breasts creating an over lab condition that must be accounted for in order to deliver therapy.

To overcome these issues the flexible removable racks that make up the application tools take on needed attributes to accommodate the patients skin contours and condition. For example, a rack made up of rows and columns may have a slit between one or more rows extending ninety percent of the length of the row. The ten percent remaining acts as a hinge, allowing the rows to be angled down when attached to avoid a fat roll or surgery scar that is healing.

Or a rack for a large breasted woman may have 4 rows of array elements followed by a 4-inch spacer, followed by 4 more rows of array elements. The top rows cover the top half of the breast, the spacers allow the remaining rows to be placed underneath the breasts. Any such adaptations can be designed into the application tools.

One summary of this invention is an apparatus for applying an alternating electric field to a subject's body, the apparatus including a plurality of sets of at least two electrode elements held in an application rack; a water-soluble medical adhesive applied to the underside of the plurality electrode elements for adhering to a patient's body, the application rack used to position the electrode elements on the patient with the medical adhesive being instrumental in adhering the electrode elements to the skin of the patient in predetermined positions, the rack then being separated from the arrays of electrodes positioned on the skin of the patient; a vented stretchable shirt or garment poisoned on the patient to provide additional contact to the plurality of electrodes thereby further securing the electrodes to the patient; and a plurality of temperature sensors, wherein each of the temperature sensors is positioned to sense a temperature at a respective set of electrode elements and generate a respective signal indicative of the sensed temperature.

The treatment is begun with real time temperature readings. At least one initial temperature is taken to establish a baseline temperature profile. The deviation of the temperature at each sensor being used to determine a temperature differential that can be used in the control scheme of firing particular sets of electrode elements 30 based on the differential. This feature allows for a self-calibration of the temperature sensors and to compensate for various temperatures on the skin of the patient. Each firing configuration is run evaluating which sequence produces the highest field intensity with the lowest duty cycle on array elements (which is understood to result in the lowest temperature rise. Heat sensors on each array element provide for real time feedback and field intensity monitoring.

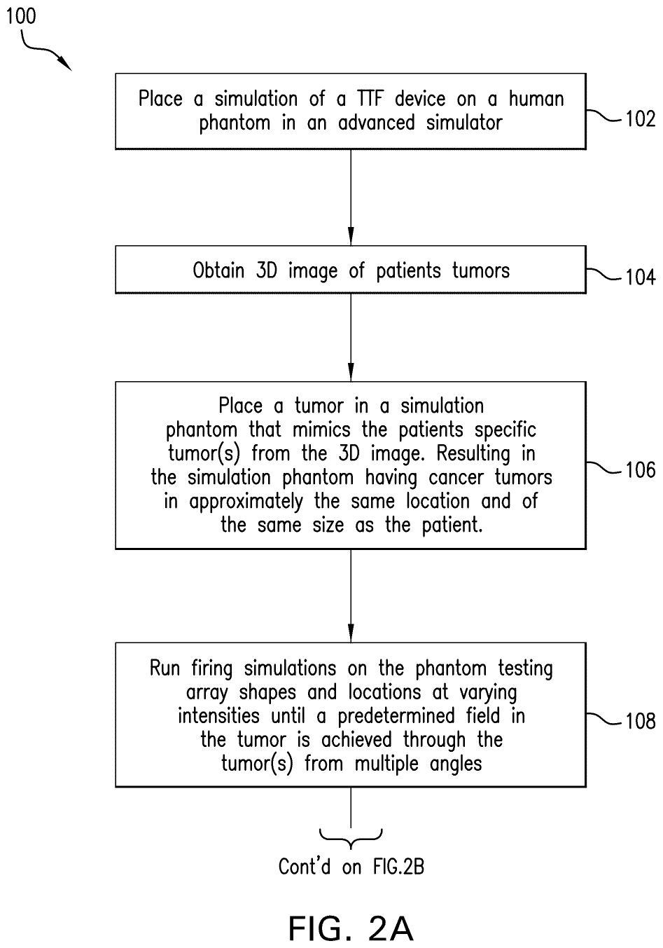

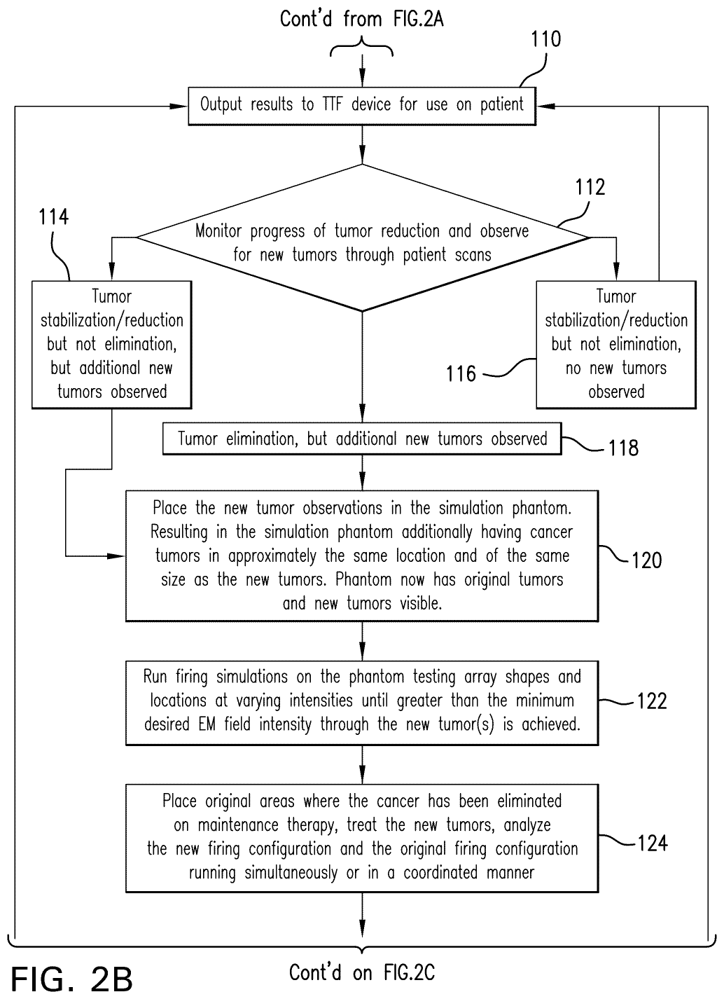

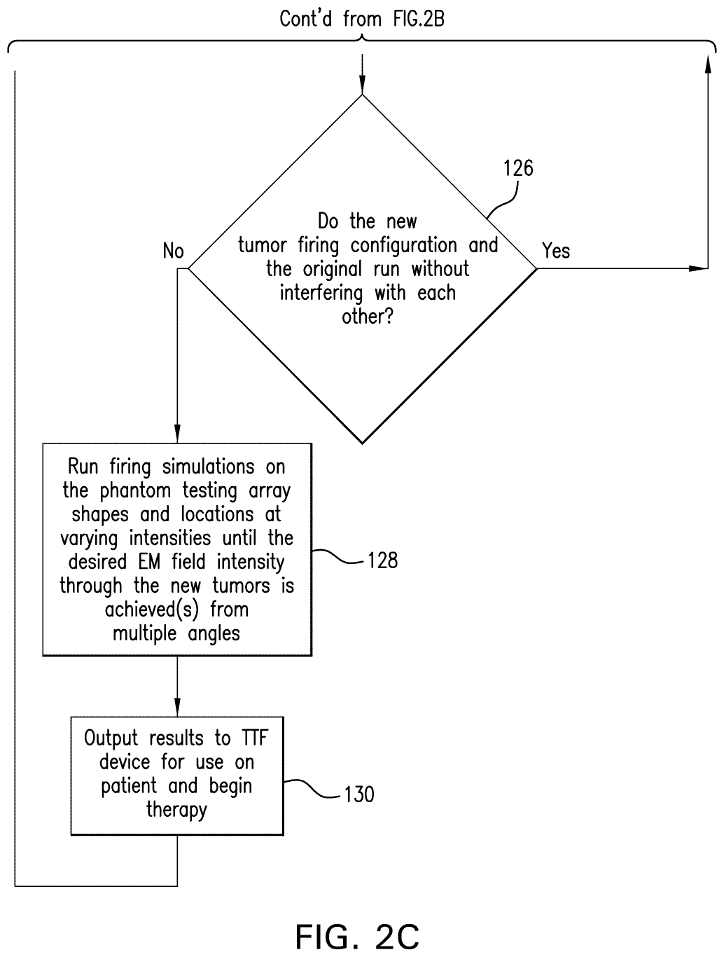

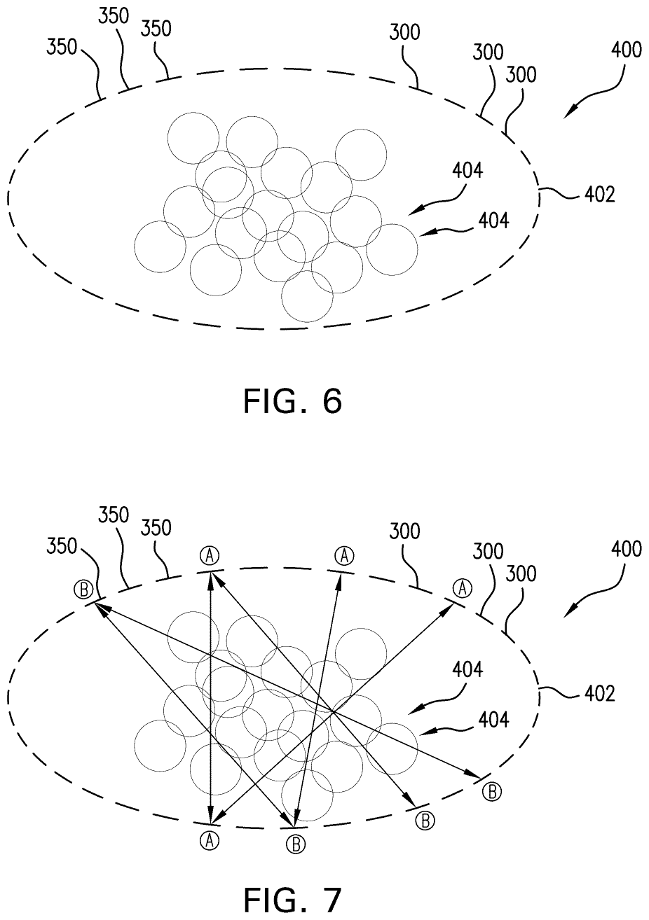

Now, additionally referring to FIGS. 2A-C there is illustrated a method 100 for applying Adaptive TTF to treat tumors. Method 100 uses a simulated human phantom to simulate the location of the tumors and to calculate the proper positions of electrode elements 30 to effectively treat the tumors and to determine the location of alternate electrode positioning to treat the tumors at differing angles and to determine the intensity levels of the alternate angles used in the treatment regime. At step 102, a simulation of TTF device 10 is utilized with phantoms of electrodes 30 placed on the phantom body in the simulator, to prepare the simulated body for simulated treatment of the simulated tumors. At step 104, a three-dimensional image of the contour of the patient's body and locations of the patient's tumors is obtained, by way of three dimensional imaging techniques. At step 106, the simulated tumors are dynamically positioned in the phantom body, a two dimensional representation being shown in FIG. 6, discussed later. The placement and sizing of tumors being positioned so that geometric models of where to optimally place primary treatment paths, as well as secondary treatment paths and other alternate treatment paths can be planned for with the placement/utilization of many of the electrode elements 30, 300, 350.

At step 108, electrode firing simulations are undertaken in the simulator on the phantom body to test the locations and needed intensities to deliver a sufficient field intensity to treat the tumors. Additionally, the determination of the frequencies of the electromagnetic fields used in the treatment are simulated to determine the variation in frequencies and intensity variations needed to supply effective treatment. The size of tumors, type of tumors and cell sizes all being part of the simulation. All being undertaken at various angles as illustrated in a two-dimensional image of FIG. 7, but being done in three dimensions in the simulator. At step 110, the results of the simulation are transferred to TTF device 10 for treatment of the patient.

At step 112, the actual path of method 100 is determined by the three results articulated in steps 114, 116 and 118. In step 112, the effectiveness of treatment is gauged by an analysis of the treated cells in the tumors. A cell analysis can include a biopsy, a scan, a three-dimensional location, a physical size or any detection technique that is used to detect the presence/absence/condition of cancer cells. If the analysis indicates that tumors are not eliminated and new tumors are observed as articulated in step 114, then the method is terminated and some other additional treatment is started. If the analysis indicates that the tumors are reduced and no new tumors are observed, as articulated in step 116, then method 100 loops back to step 110 and treatment continues. If the analysis indicates that tumors are eliminated, but new tumors are observed, as articulated in step 118, then method 100 proceeds to step 120.

At step 120, the analysis that has taken place in steps (or blocks) 112, 114, 116 and 118 has resulted in the observation of new tumors. The locations and characteristics of these new tumors are input into the phantom model so that the model includes the previously detected tumors, and the newly detected tumors all positioned in the three-dimensional model. At step 122, new electrode element 30 firing configurations are run to determine the geometrical configuration on the phantom body surface of the positions of electrode elements 30 in order to achieve the desired electromagnetic field intensity in the locations of the tumors. At step 124, the areas where tumors have been eliminated are placed on a maintenance therapy level of ATTF intensity, while the new tumor positioned are selected for treatment therapy levels. At decision step 126, it is determined whether the new firing configuration will run without interfering with the previous firing configuration. If the answer is yes, then method 100 proceeds back to step 110. If the answer is no, then method 100 proceeds to step 128.

At step 128, the firing configuration is altered and tested until the subarray shapes and locations achieve the needed electromagnetic field strengths in the new tumor locations. At step 130, the developed firing configuration is then transferred to TTF device 10 for patient treatment, and method 100 proceed back to step 110.

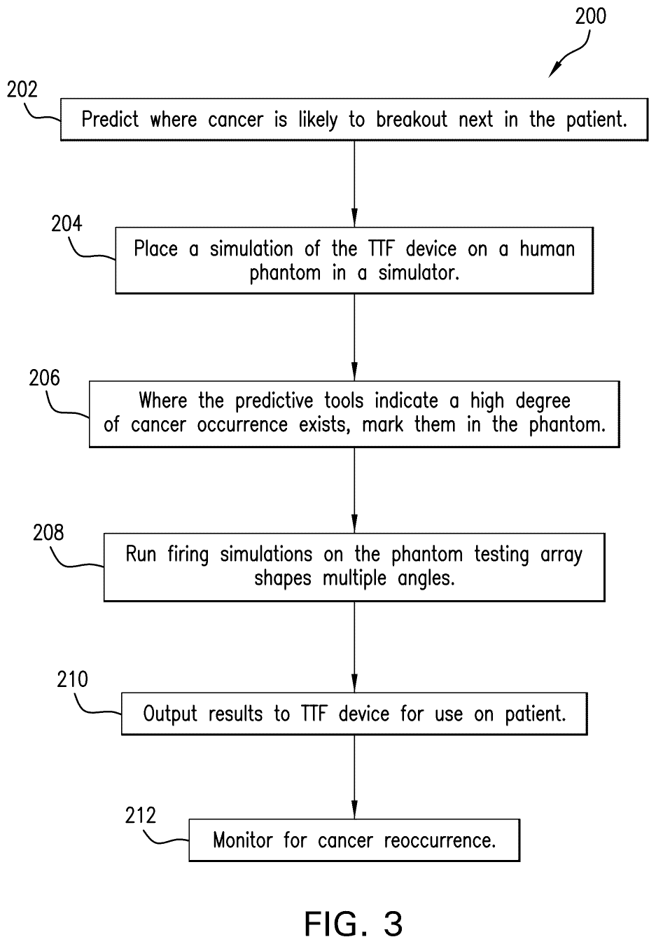

Now, additionally referring to FIG. 3, another inventive method of the present invention is illustrated as method 200. At step 202, method 200 predictive tools are used to determine where new cancer growth is likely to occur. At step 204, a simulation of TTF device 10 is, in a phantom manner, placed on a human phantom in the simulator. This includes the geometrical configuration of subarrays of electrodes 30 on the surface of the phantom human body. At step 206, the predicted locations of new tumors are positioned in the human phantom. At step 208, electrode firing configurations are developed for the existing and predicted tumors, with the firing configurations being arranged to take place at multiple angles in the phantom using selected subarrays of electrode elements 30.

These developed firing configurations are then output to TTF device 10 at step 210, for use on the patient. As discussed in method 100, analysis of the cancer treatment is undertaken, and any reoccurrence of cancer is monitored in step 212.

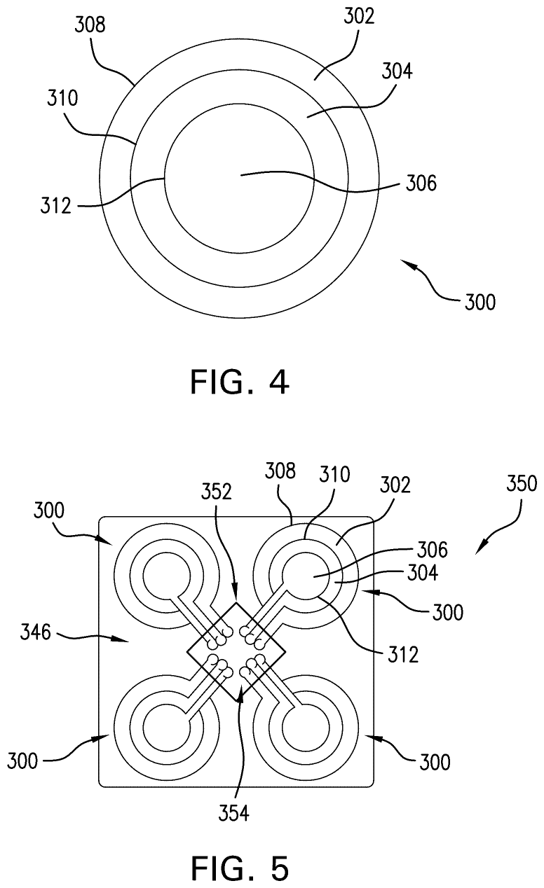

Now, additionally referring to FIG. 4, there is illustrated a new inventive design for an electrode 30 in the form of electrode element 300. Electrode element 300 has been illustrated as three concentric electrodes 302, 304 and 306, with an outer circumference 308. Electrodes 302 and 304 are ring shaped and are separated by an insulating material or spacing 310. In a like manner electrode 304 is electrically isolated from electrode 306 by an insulating material or spacing 312. Each electrode element 302, 304 and 306, while a part of electrode 300, are separately accessible by TTF device 10, so that anyone or a combination of elements are activated during the firing of electrode 300. An advantage of this allows for variations in the heat distribution inherent in the use of electrode 300. Another advantage is that since the edges of electrodes may have an abnormally high level of electrical charge, having a longer effective circumference distributes the charge so that it is less concentrated. While FIG. 4 is illustrated as three concentrically formed elements, other shapes and numbers of segmented electrodes are contemplated. For example, an electrode with an outer perimeter of a rectangular shape having segmented electrode elements that are angularly divided radially outward from a center, would be one example of the segmented electrodes.

Now, additionally referring to FIG. 5, there is shown a singular electrode assembly 350 having four segmented electrodes 300 positioned on an application element 346, where a controller 354 is located on a footprint 352. Controller 354 is illustrated as being electrically connected to each of the three segmented electrodes in each of electrodes 300 by way of electrical connections extending from each segmented electrode of each electrode 300. It is also contemplated that application element 346 itself may be a separate electrode. It is further contemplated that other geometric shapes can exist on application element 346.

Now, additionally referring to FIG. 6, there is shown in a two-dimensional cross-sectional representation of a human body 400 with multiple tumors 404 positioned therein. The seeming overlapping of tumors 404, is a way of discussing that the three-dimensional aspects, discussed herein, are considered in the carrying out of the methods of the present invention. On a surface 402 of the human body, electrodes 300, 350 are positioned as determined in the simulations discussed herein.

Now, additionally referring to FIG. 7, there is shown the human body 400 during an execution of an illustrated firing configuration. While this representation shows several lines extending from the circled-A and circled-B electrodes that intersect, it is to be understood that these lines actually exist in three dimensions and are not likely intersecting. First, considering the lines extending between circled-A's, this represents a first firing configuration, where the TTFs are applied to those electrodes causing the TTFs to extend from one circled-A to another circled-A. The next step in the firing configuration would be between circled-B's. Another step in the firing configuration is between some circled-A's and circled-B's. This illustrates the individual electrode selection so that differing electrodes 300, 350 are operating in alternating subarrays of electrodes. Another value of this illustration is that if a particularly difficult or long path is selected, and it is not normally possible to use this particular path with the normal intensity filed, then individual electrodes, say the two circled-B electrodes with the longest path, can be activated with opposite phases of the normal signal thereby effectively doubling the field strength at the target.

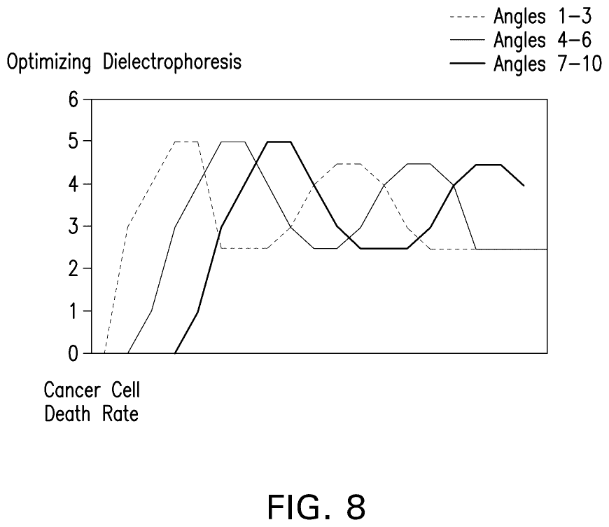

Now, additionally referring to FIG. 8 there is shown a cancer treatment effectiveness chart, where ten sets of angles are utilized in the treatment of the tumors. Angles 1-3, 4-6 and 7-10 are used in rotation to keep the level of dielectrophoresis at a high level. As can be seen when using any one set of angles the effectiveness reduces after reaching the level 5. However, by time phasing the introduction of other angle sets, during treatment, the effectiveness has a longer run of level 5, then level 4, thus enhancing the overall death rate of cancer cells in the tumors. It is thought that the various orientations of the genetic material in the dividing cancer cells are at certain angles more or less vulnerable to the TTFs, so that varying the angles effectively allows fewer cells to escape the EM coupling of the treatment fields. This changing and rotating of angular treatments fields serves to enhance the effectiveness of TTF treatment.

In a further embodiment of the present invention, a monitoring system of the effectiveness of treatment, during treatment is now discussed. The present invention senses a signal from the oscillating genetic material of the destructing cells. The genetic material during reproduction takes on an electrical charge, which during TTF treatment becomes coupled to the EM field. This coupling causes physical movement due to its electrical charge to thereby destroy the EM coupled material. As the physical oscillating electrical charge of the genetic material moves it creates a small EM field of its own that is at exactly the frequency of its physical movement. These small created EM fields are detectable and show up as slight frequency differences or a generated harmonic frequency. These created EM fields, when detected, provide information as to how effective the input field is coupling to the charged cell portion. This detection paves the way for several feedback methods to alter the input field direction, intensity, and enables a real time measure of the efficacy of treatment.

The EM signature relates to how the material is destructing. If the genetic material elongates or shortens during destruction, then during the period of destruction the created EM field reflects the change in the physical movements during destruction (and hence a frequency shift). A receiver is added to the TTF device 10 to detect and alter the function of TTF device 10. The receiver has a receiving bandwidth that receives the created signal which appears as a small wave packet. As more material is destructing the signal packets increase, if the destruction of cells is on the decline then the detected EM signal bursts decline.

Treatment safety is assessed by reporting adverse events, effectiveness is assessed by measuring blood tumor markers and computerized tomography (CT) data to determine if the disease is progressing, is static, or is regressing.

This therapy is initially assessed as a monotherapy; therefore subjects will not be on chemotherapy, radiation, or other standards of care during an initial treatment. If the monotherapy is determined that a subject is benefiting from this therapy, the patient will continue with this therapy alone. If it is determined that the patient is not benefiting from this therapy, the patient will discontinue the monotherapy and be treated with standard of care therapies.

One aspect of the present invention is to evaluate the safety and efficacy of Adaptive TTF (ATTF), in patients with recurring asymptomatic metastatic disease in the torso with cancers that have cell sizes between 3 and 20 μm. For example, an open label, interventional pilot trial using a single arm of treatment can be undertaken as now discussed. The study begins with delivering ATTF therapy for a 4-week period. At the start of the study, the size of a patient's cancer cells is determined based on what TTF frequency has safely treated the cancer in previously run human trials. For example, breast cancer, non-small-cell lung cancer (NSCLC), pancreatic cancer, and liver cancer would be classified as average to large on a scale of 3 to 20 μm. Based on human trials already run, the frequency of 150 kHz will be utilized. Ovarian cancer would be classified on the small end of the scale and as done in previous studies will be treated at 200 kHz.

It should be emphasized here that even though TTF therapy has been shown effective in frequency ranges of between 100 kHz and 300 kHz, in pre-clinical research, our study design is deliberately restricting its initial frequencies to only those that have been proven safe in human trials. Furthermore, after the initial therapy, if required our adaptations explained below are restricted to between 100 kHz and 250 kHz to more closely reflect those frequencies already shown safe in human trials.

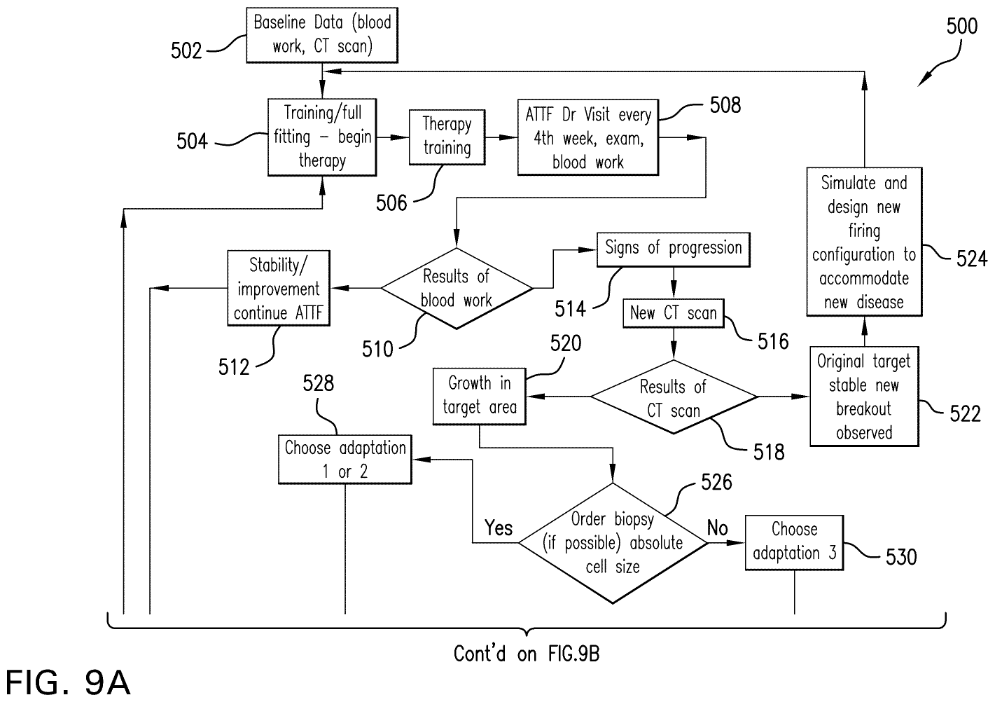

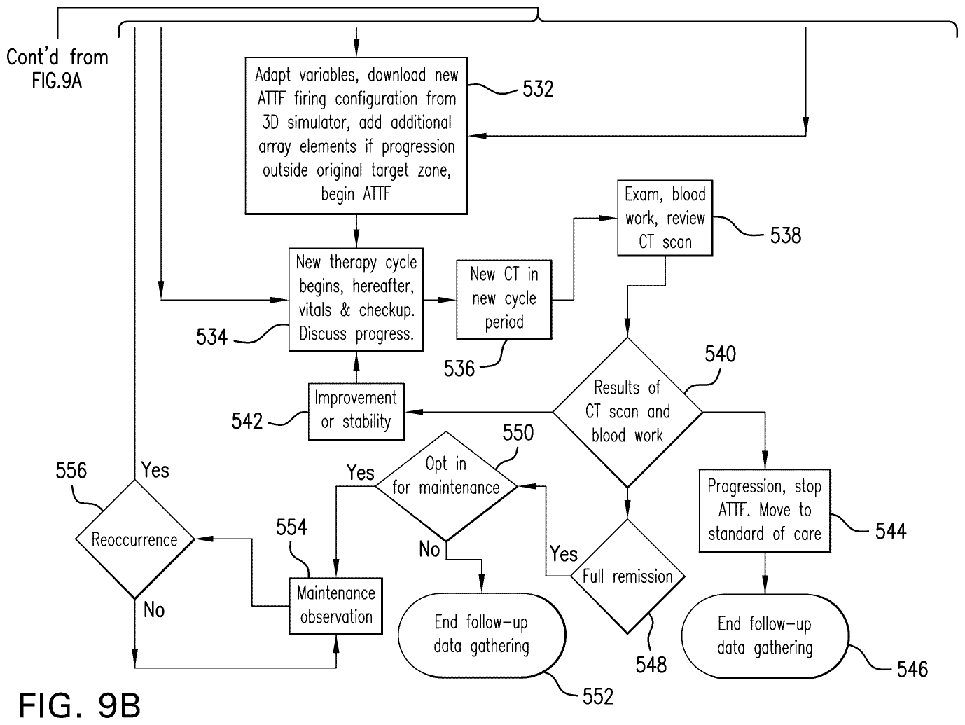

Now, additionally referring to FIGS. 9A and 9B, a method 500 that is useful for both trial and treatment applications of ATTF treatments of tumors is discussed. At step 502, a CT scan is taken of each patient along with blood work to establish baseline data. The CT scan will be imported into a 3D simulator for the purpose of producing possible firing configurations for ATTF. The 3D simulations with CT scan will be used to make recommendations of firing configurations to the doctor.

At steps 504 and 506 training is undertaken so that the patient will receive effective care. The doctor will conduct a physical exam of the patient while firing configurations are being simulated on the patient through LED light ups of array elements. This will be accomplished with the help of a trained technician. The doctor will then use the recommendations and live simulations to determine the optimal firing configuration for ATTF. At step 508, the doctor periodically visits the patient to determine the responsiveness of the cancer treatment. Blood work is conducted on a blood sample from the patient. The results of the blood test at step 510 are included in boxes 512 and 514. At 512 if stability and patient improvement occur, then method 500 proceeds to step 534. At 514, if there are signs of the cancer progressing, then method 500 proceeds to step 516.

Optimization is determined through the beneficial manipulation of intensity, frequency and of the number of strategic angles of delivery to 3 or more. Final firing configurations will target the metastatic disease. In addition, when appropriate, the primary or previously active area of disease will be targeted simultaneously. For example, if pancreatic cancer has spread to the liver both the liver and pancreas will be treated. If a new second area of disease appears while reduction of disease in the original target area occurs, it will also be treated simultaneously. All patients included in this trial must be diagnosed with reoccurring asymptomatic metastatic disease from cancers that have been established as having typical tumor cell radiuses of between 3 and 20 (μm). The following types of cancers are some examples of cancers that normally have the targeted cell size: Liver, stomach, pancreatic, colon, mesothelioma, non-small cell lung, ovarian, cervical, breast, and melanoma.

Eligible patients will be enrolled, baseline tests will be performed, and baseline data gathered. The patients will be treated continuously with the device for a period of 4 weeks at which time a doctor's exam and new blood work will be performed. At step 516 of method 500, if new blood work indicates possible progression a new CT scan will be performed. If there is a response to the therapy in the form of reduced disease in the target area the therapy will continue for an additional 4 weeks, at step 512. If there is a development of disease outside the target area additional arrays 32, 34 may be added to target the new area of disease.

At step 518, if the CT scan shows growth in the target area (block 520), then the method proceeds to step 526 or shows a new breakout (block 522), then the method proceeds to step 524. At step 524, new firing configurations are arranged to accommodate the appearance of new tumors, and then the method returns to step 504 as needed. If after the first 4 weeks the blood work, doctor's exam, and CT scan show disease stability or progression the doctor will, if possible, order a biopsy, at step 526, after 24 hours of therapy cessation. The absolute cell size of the tumor will be determined from the biopsy. If there has been no significant cell size change (proceed to step 530) from the expected range the following adaptation will be implemented.

It is known that while TTF therapy is active it can temporarily and intermittently alter cells size to a larger size because of its disruption of septum formation. When therapy is stopped, and the cancer cells are no longer under TTF therapy they return to their normal size. As mentioned above, it has also been documented that the enlargement of cell size caused by TTF therapy can cause some tumor cells to escape the antimitotic effect of TTF. Research indicates that this escaping due to cell size change can be compensated for by alternating between the original frequency and a lower frequency. The lower frequency effectively treats the enlarged cells because of the inverse relationship between cells size and optimal frequency. For example, Ovarian cancer treated at 200 kHz would be rotated with a frequency of 150 kHz. Rotating of the frequencies means a use of one frequency for a preselected time, then the use of an alternate frequency for another preselected time. The rotating of electromagnetic field frequencies means the selection of a frequency within the range of frequencies for a selected time and then selecting another frequency within the range, continuing on in this manner selecting new frequencies within the range. In this case no frequency outside the range of 100 kHz to 250 kHz will be used in this adaptation. Implementing this strategy is referred to as ADAPTATION 1 (step 528).

If the results of the biopsy show a significant size change from the expected range, it is likely that previous treatments or TTF therapy itself have killed cells of the expected size range and outlier cells of a different size have begun to dominate. In this situation the doctor will order a frequency range adjustment to most closely match the optimal frequency for the new cell size. No frequency outside 100 kHz to 250 kHz will be used in this adaptation, which is referred to as ADAPTATION 2 (step 528).

If a scan of the tumors does reveal only stability or worsening and a biopsy is deemed too difficult or risky, and therefore is not completed, the physician is left with no indication or confirmation of cell size, in this situation the following adaptation will be implemented. A frequency rotation sequence will begin based on the type of cancer. For example, metastatic Pancreatic cancer with a historical target frequency of 150 kHz, may now be treated at 175 kHz, then 150 kHz, and then 125 kHz in rotation. This allows for a logical adjustment in frequency when tumor cells size may not be known. Sequences between 100 kHz and 250 kHz will be used at 0.6 (or longer) second intervals and the interval may be limited to 3 seconds or less. This is referred to as ADAPTATION 3 (step 530).

The doctor may also order additional adaptations of therapy based on ATTF variables, e.g., increased intensity, additional angles, etc. These non-frequency adaptations can be added to any of the above adaptations. The justifications for the non-frequency adaptations are multiple. Some tumors may be gone, allowing new angle optimization on remaining tumors. There may be mixed responses where overall tumors are reduced, but some tumors on the edge of a TTF may not be affected. Adjusting angles will compensate for this phenomenon, etc.

We theorize the above adaptations and request for biopsy are justified. The reason being that in all the references in this document, and now in the dozens of peer reviewed articles published in scientific journals there has not been one instance where TTF therapy has not reduced tumors. However, this effectiveness is dependent on the frequency being optimally matched to the cell size of the tumor, the intensity of the TTField being at the effective level and the TTField being applied from enough angles.

At step 532, new ATTF firing configurations from simulations, discussed herein, are used to target the new tumors. At step 534, the therapy continues for 4 additional weeks. At the end of the second 4 weeks of therapy (steps 536 and 538) new blood work, CT scans and doctor's exam will be ordered. If positive response (block 536 from decision 540) is observed the therapy will continue for an additional 4 weeks (returning to step 534). At step 544, if there is a newly developed disease outside the target area additional arrays may be added to target the new area of disease, or move to a standard of care, and an end at step 546. If the second 4 weeks of therapy shows progressive disease the doctor and patient will decide whether a phenomenon known as pseudo progression is at play. This is a condition where dying cancer cells swell up in the process, appearing as growth on a scan only to later die and be absorbed by the body. If Pseudo progression is suspected the doctor and patient may elect to continue therapy for a 3^{rd} month. If stability or regression are not seen after 3 months, ATTF will cease, and a standard of care treatment will begin.

Patients showing positive results will continue on ATTF until full disease remission (step 548) at which time they may choose to end participation in the study or continue for the long-term therapy phase of the study (Steps 550-556).

The long-term maintenance protocol (steps 550, 554, 556) will consist of close monitoring for signs of re-occurrence through tumor markers, and image scans. If re-occurrence is found, immediate application of ATTF will resume according to the original protocol. Any patient who develops toxicity to ATTF (skin irritation) that is deemed to be unmanageable will cease therapy and continue to standard of care chemotherapy.

Treatment consists of wearing electrically insulated electrode arrays on and around the circumference of the torso or disease area. Electrode array placement may require shaving the abdomen/back before and during treatment. After an initial passive test that includes the wearing of the device and a short visit to the clinic for training and monitoring, patients will be released to continue treatment at home. There they will be visited by nurses for follow-up training on maintaining their regular daily routine. In addition, patients will be contacted once per month by telephone to answer basic questions about their health status.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A method of adapting a treatment of tumors by the delivery of tumor treating electromagnetic fields to a patient, comprising the steps of:
placing an electrode array on the patient;
selecting a first adaptation of an electrode activation configuration to activate selected elements of the electrode array to treat the tumors;
analyzing cells of the tumors that have been treated using the first adaptation of the electrode activation configuration; and
using the first adaptation of the electrode activation configuration to treat the tumors until depending on a cell analysis of the analyzing step then changing to a second adaptation of the electrode activation configuration to activate selected elements of the electrode array to treat the tumors, the first adaptation including a rotating of electromagnetic field frequencies within a range of frequencies of between 200 kHz and 150 kHz.

2. The method of claim 1, further comprising the step of simulating a new firing configuration of the electrodes to be included in the second adaptation of the electrode activation configuration.

3. The method of claim 2, wherein the second adaptation of the electrode activation configuration includes using TTF frequencies between 100 kHz and 250 kHz.

4. The method of claim 2, further comprising a step of simulating a new firing configuration of the electrodes to be included in a third adaptation of the electrode activation configuration.

5. The method of claim 4, wherein the third adaptation of the electrode activation configuration includes rotating TTF frequencies between 150 kHz, then 175 kHz, then 150 kHz, then 125 kHz, then back to 150 kHz in repeating fashion.

6. The method of claim 4, wherein the third adaptation of the electrode activation configuration includes varying TTF frequencies between selections between 100 kHz and 250 kHz.

7. The method of claim 6, wherein each frequency selected will be used for a duration of at least 0.6 seconds.

8. The method of claim 7, wherein the selected duration is less than 3 seconds.

9. A method of treating of tumors by the delivery of tumor treating electromagnetic fields to a patient, comprising the steps of:
placing at least one electrode array on the patient;
selecting a first adaptation of an electrode activation configuration to activate selected elements of the electrode array to treat the tumors;
analyzing cells of the tumors that have been treated using the first adaptation of the electrode activation configuration; and
using the first adaptation of the electrode activation configuration to treat the tumors until, depending on a cell analysis of the analyzing step, then changing to a second adaptation of the electrode activation configuration to activate selected elements of the electrode array to treat the tumors.

10. The method of claim 9, further comprising the step of simulating a new firing configuration of the electrodes to be included in the second adaptation of the electrode activation configuration.

11. The method of claim 10, wherein the second adaptation of the electrode activation configuration includes using TTF frequencies between 100 kHz and 250 kHz.

12. The method of claim 10, further comprising a step of simulating a new firing configuration of the electrodes to be included in a third adaptation of the electrode activation configuration.

13. The method of claim 12, wherein the third adaptation of the electrode activation configuration includes rotating TTF frequencies between 150 kHz, then 175 kHz, then 150 kHz, then 125 kHz, then back to 150 kHz in repeating fashion.

14. The method of claim 12, wherein the third adaptation of the electrode activation configuration includes varying TTF frequencies between selections between 100 kHz and 250 kHz.

15. The method of claim 14, wherein each frequency selected will be used for a duration of at least 0.6 second intervals.

16. The method of claim 15, wherein the selected duration is less than 3 seconds.

17. A method of treating tumors by the delivery of tumor treating electric fields to a patient, comprising the steps of:
importing a medical image of the patient into a medical simulator;
placing at least one electrode array on the patient;
using the medical simulator to simulate a firing configuration of the electrodes to be included in adaptations of the electrode activation configuration;
selecting a first adaptation of an electrode activation configuration to activate selected elements of the electrode array to treat the tumors, the first adaptation of an electrode activation configuration including the firing configuration of a first adaptation of the electrode activation configuration;
analyzing cells of the tumors that have been treated using the first adaptation of the electrode activation configuration; and
using the first adaptation of the electrode activation configuration to treat the tumors until depending on a cell analysis of the analyzing step then changing to a second adaptation of the electrode activation configuration to activate selected elements of the electrode array to treat the tumors.

18. The method of claim 17, further comprising the step of simulating a new firing configuration of the electrodes to be included in the second adaptation of the electrode activation configuration.

19. The method of claim 18, wherein the second adaptation of the electrode activation configuration includes using TTF frequencies between 100 kHz and 250 kHz.

20. The method of claim 18, further comprising a step of simulating a new firing configuration of the electrodes to be included in a third adaptation of the electrode activation configuration.
